(19) United States
(12) Patent Application Publication
(10) Pub. No.: US 2024/0010970 A1
(43) Pub. Date: Jan. 11, 2024

(54) ANTIMICROBIAL BIOSENSORS

(71) Applicant: The Jackson Laboratory, Bar Harbor, ME (US)

(72) Inventor: Julia Oh, Bar Harbor, ME (US)

(73) Assignee: The Jackson Laboratory, Bar Harbor, ME (US)

(21) Appl. No.: 18/032,845

(22) PCT Filed: Oct. 20, 2021
(86) PCT No.: PCT/US2021/055744

§ 371 Date: Apr. 20, 2023

Related U.S. Application Data
(60) Provisional application No. 63/094,858, filed on Oct. 21, 2020.

(51) Int. Cl. C12N 1/20; A61K 38/17; A61P 31/00; G01N 33/569

(52) U.S. Cl. CPC C12N 1/20; A61K 38/1729; A61P 31/00; G01N 33/569; G01N 2800/26

(57) ABSTRACT

Provided herein are antimicrobial biosensors for skin for treating Staphylococcus aureus Type I, Type II, Type III, and/or Type IV infection. In some embodiments, the antimicrobial biosensors are expressed in Staphylococcus epidermidis cells.

### RELATED APPLICATIONS

This application claims the benefit under 35 U.S.C. § 119(e) of U.S. provisional application No. 63/094,858, filed Oct. 21, 2020, which is incorporated by reference herein in its entirety.

### GOVERNMENT LICENSE RIGHTS

This invention was made with government support under W81XWH181 0229 awarded by Department of Defense. The government has certain rights in the invention.

### BACKGROUND

The occurrence and virulence of pathogens such as methicillin-resistant Staphylococcus (S.) aureus (MRSA) is rapidly increasing, with a limited number of antibiotics available or in development to treat infections, especially those associated with skin. There is significant need for new therapeutics for prevention as well as acute care.

A potential limitation of existing antimicrobials is that they are typically broad spectrum and can significantly impact the surrounding skin microbiome, the diversity of which has been shown to improve cutaneous health, resist pathogen colonization, and bolster host immune response to pathogens. In addition, increasing use may increase relative risk for development and spread of antibiotic resistance.

### SUMMARY

The present disclosure provides, in some aspects, microbial therapeutic biosensors for skin that provide spatial and temporal control of antimicrobial exposure. The use of an engineered microbe design to deliver an antimicrobial agent in a controlled manner, as described herein, provides key benefits over using naturally-occurring antimicrobial-secreting probiotic strains. For example, an engineered commensal can be modified to have desirable characteristics that improve its colonization, longevity, and antimicrobial properties versus any inherent limitations to the probiotic, which may have limited or differential colonization success in the skin. As the union of the concept of a targeted, engineered, on-demand antimicrobial therapeutic, the biosensor organism can detect the presence of a pathogenic bacteria (e.g., S. aureus) and respond accordingly, producing a suite of different human-derived or bacteria-derived antimicrobials.

Some aspects of the present disclosure provide an engineered Staphylococcus (S.) epidermidis cell comprising an S. aureus quorum-sensing receptor. In some embodiments, the S. aureus quorum-sensing receptor is a component of an S. aureus quorum-sensing signal-response system.

In some embodiments, the cell is an agr deficient mutant S. epidermidis cell.

In some embodiments, the quorum-sensing signal-response system comprises a first promoter sequence operably linked to a therapeutic peptide coding sequence.

In some embodiments, the promoter sequence is activated by signaling of the quorum-sensing signal-response system.

In some embodiments, the therapeutic peptide coding sequence is an antimicrobial peptide coding sequence.

In some embodiments, the quorum-sensing signal-response system comprises (a) an S. aureus agrA coding sequence encoding AgrA and (b) an S. aureus agrC coding sequence encoding S. aureus AgrC.

In some embodiments, the first promoter sequence is activated by S. aureus AgrA.

In some embodiments, the S. aureus agrA coding sequence is operably linked to a second promoter sequence.

In some embodiments, the S. aureus agrC coding sequence is operably linked to a second promoter sequence.

In some embodiments, the S. aureus agrA coding sequence and the S. aureus agrC coding sequence are operably linked to a second promoter sequence.

In some embodiments, the second promoter sequence is a constitutive promoter sequence.

In some embodiments, the first promoter sequence and second promoter sequence are oriented in opposite directions relative to each other.

In some embodiments, the first promoter sequence and the second promoter sequence are components of a bidirectional promoter.

Other aspects of the present disclosure provide engineered agr deficient mutant Staphylococcus (S.) epidermidis cell comprising a nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial peptide coding sequence, wherein the bidirectional promoter is activated by an S. aureus AgrA protein.

In some embodiments, the size of the antimicrobial peptide is 5 kD to 100 kD, or 5 kD to 50 kD.

In some embodiments, the antimicrobial peptide is LL-37. In some embodiments, the antimicrobial peptide is elafin. In some embodiments, the antimicrobial peptide is hiracin.

Yet other aspects of the present disclosure provide an engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial LL-37 peptide coding sequence, wherein the bidirectional promoter is activated by an S. aureus AgrA protein.

Still other aspects of the present disclosure provide an engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial elafin peptide coding sequence, wherein the bidirectional promoter is activated by an S. aureus AgrA protein.

Some aspects of the present disclosure provide an engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial hiracin peptide coding sequence, wherein the bidirectional promoter is activated by an S. aureus AgrA protein.

In some embodiments, the bidirectional promoter comprises a constitutive promoter sequence, optionally an S. epidermidis promoter sequence, and an S. aureus P3 promoter sequence, or an S. aureus P2 promoter sequence and an S. aureus P3 promoter sequence.

In some embodiments, the constitutive promoter sequence or the S. aureus P2 promoter sequence is operably linked to the S. aureus agrA coding sequence and the S. aureus agrC coding sequence, and the S. aureus P3 promoter sequence is operably linked to the antimicrobial peptide coding sequence.

In some embodiments, the S. aureus agrA coding sequence and the S. aureus agrC coding sequence are classified as S. aureus Type I, Type II, Type III, or Type IV coding sequences.

In some embodiments, the S. aureus agrA coding sequence and the S. aureus agrC coding sequence are classified as S. aureus Type I coding sequences.

In some embodiments, the S. epidermidis cell is classified as strain Tu3298 or an agr deficient mutant thereof.

In some embodiments, the engineered nucleic acid is integrated into the S. epidermidis cell genome.

In some embodiments, the S. epidermidis cell genome includes a single copy of the engineered nucleic acid.

In some embodiments, the S. aureus agrA coding sequence, the S. aureus agrC coding sequence, and/or the antimicrobial peptide coding sequence is codon optimized for expression in S. epidermidis.

In some embodiments, the cell is immune to the antimicrobial peptide.

In some embodiments, the cell comprises a gene the confers resistance to the antimicrobial peptide.

In some embodiments, the antimicrobial peptide is linked to a secretion signal sequence.

Some aspects of the present disclosure provide a composition comprising the engineered cell of any one of the preceding paragraphs.

In some embodiments, the composition is formulated for topical administration. For example, the composition may be formulated as a biofilm, a cream, a lotion, or a gel.

Other aspects of the present disclosure provide an engineered nucleic acid comprising (a) a first promoter sequence operably linked to a LL-37 coding sequence and (b) a second promoter sequence operably linked to an S. aureus agrA coding sequence and an S. aureus agrC coding sequence, wherein the first promoter is activated by an S. aureus AgrA protein.

Still other aspects of the present disclosure provide an engineered nucleic acid comprising (a) a first promoter sequence operably linked to an elafin coding sequence and (b) a second promoter sequence operably linked to an S. aureus agrA coding sequence and an S. aureus agrC coding sequence, wherein the first promoter is activated by an S. aureus AgrA protein.

Further aspects of the present disclosure provide an engineered nucleic acid comprising (a) a first promoter sequence operably linked to a hiracin coding sequence and (b) a second promoter sequence operably linked to an S. aureus agrA coding sequence and an S. aureus agrC coding sequence, wherein the first promoter is activated by an S. aureus AgrA protein.

In some embodiments, the first promoter sequence and second promoter sequence are oriented in opposite directions relative to each other.

In some embodiments, the first promoter sequence and the second promoter sequence are components of a bidirectional promoter.

Some aspects of the present disclosure provide composition comprising two cells, a first cell and a second cell, of any one of the preceding claims, wherein the S. aureus quorum-sensing signal-response system of the first cell is of a first strain, and the S. aureus quorum-sensing signal-response system of the second cell is of a second strain.

In some embodiments, the first strain and the second strain are selected from a Type I S. aureus strain, a Type II S. aureus strain, a Type III S. aureus strain, and a Type IV S. aureus strain.

In some embodiments, (a) the first strain is a Type I S. aureus strain, and the second strain is a Type II S. aureus strain; (b) the first strain is a Type I S. aureus strain, and the second strain is a Type III S. aureus strain; (c) the first strain is a Type I S. aureus strain, and the second strain is a Type IV S. aureus strain; (d) the first strain is a Type II S. aureus strain, and the second strain is a Type III S. aureus strain; (e) the first strain is a Type II S. aureus strain, and the second strain is a Type IV S. aureus strain; or (f) the first strain is a Type III S. aureus strain, and the second strain is a Type IV S. aureus strain.

Other aspects of the present disclosure provide a composition comprising three cells, a first cell, a second cell, and a third cell, of any one of the preceding claims, wherein the S. aureus quorum-sensing signal-response system of the first cell is of a first strain, the S. aureus quorum-sensing signal-response system of the second cell is of a second strain, and the S. aureus quorum-sensing signal-response system of the third cell is of a third strain.

In some embodiments, the first strain, the second strain, and the third strain are selected from a Type I S. aureus strain, a Type II S. aureus strain, a Type III S. aureus strain, and a Type IV S. aureus strain.

In some embodiments, the first strain is a Type I S. aureus strain and (a) the second strain is a Type II S. aureus strain, and the third strain is a Type III S. aureus strain; (b) the second strain is a Type II S. aureus strain, and the third strain is a Type IV S. aureus strain; or (c) the second strain is a Type III S. aureus strain, and the third strain is a Type IV S. aureus strain.

Yet other aspects of the present disclosure provide a composition comprising four cells, a first cell, a second cell, a third cell, and a fourth cell, of any one of the preceding claims, wherein the S. aureus quorum-sensing signal-response system of the first cell is of a first strain, the S. aureus quorum-sensing signal-response system of the second cell is of a second strain, the S. aureus quorum-sensing signal-response system of the third cell is of a third strain, and the S. aureus quorum-sensing signal-response system of the fourth cell is of a fourth strain,

In some embodiments, the first strain, the second strain, the third strain, and the fourth strain are selected from a Type I S. aureus strain, a Type II S. aureus strain, a Type III S. aureus strain, and a Type IV S. aureus strain, and each cell is of a different strain relative to each other.

Further aspects of the present disclosure provide method, comprising administering to a subject the composition of any one of the preceding paragraphs.

In some embodiments, the subject has a virulent bacterial infection. For example, the virulent bacterial infection may be a bacterial skin infection. In some embodiments, the virulent bacterial infection is a Methicillin-resistant Staphylococcus aureus (MRSA) infection.

### BRIEF DESCRIPTION OF THE DRAWINGS

FIGS. 1A-1C. Biosensor design. (FIG. 1A) Phylogenetic tree of select staphylococci with agr groups and autoinducer structures for each of the agr types (I-IV) are shown. SEQ ID NO: 3 is an autoinducer peptide sequence for an agr type I group (AIP-1). SEQ ID NO: 4 is an autoinducer peptide sequence for an agr type IV group (AIP-2). SEQ ID NO: 5 is an autoinducer peptide sequence for an agr type III group (AIP-3). SEQ ID NO: 6 is an autoinducer peptide sequence for an agr type II group (AIP-4). (FIG. 1B) Scheme for biosensor constructs using agr components from the 4 types as a single-component system. (FIG. 1C) Example of an antimicrobial peptide (LL-37) under biosensor control.

FIGS. 2A-2B. Biosensor validation. (FIG. 2A) Type I S. aureus (Type 1 USA300), Type II S. aureus (Type 2.NR-46204), Type III S. aureus (Type 3.NR-46081), Type IV S. aureus (Type4.NR-45944), or media control (TSB) supernatant were added to S. epidermidis Ti3298 cells expressing Type I, Type II, Type III, Type IV, or control plasmid (pCN34) biosensors and GFP fluorescence produced by the biosensors was measured (OD Normalized GFP Fluorescence). (FIG. 2B) Type I biosensor was integrated into S. epidermidis strain NRLL and activation of the Type I biosensor was measured with different dilutions of Type I S. aureus supernatant (1:10, 1:20, 1:40, 1:80, 1:160) or media control (TSB) versus the parent S. epidermidis NRLL strain.

FIG. 3. In vivo biosensor validation. Germ-free C57BL6/J mice were wounded and treated with (1) 10⁵ USA300 (“USA300,” methicillin-resistant S. aureus agr type 1 strain)+10⁸ S. epidermidis (“S. epi”) Tu3298Δagr (Group 1; USA300+control S. epidermidis) or (2) 10⁵ USA300+10⁸ S. epidermidis lysostaphin-producing biosensor (Group 2; USA300+biosensor S. epidermidis). Colonized bacteria plated were plated on mannitol salt agar and S. aureus and S. epidermidis colonies were quantified (colony forming units). n=4 for Group 1; n=5 for Group 2.

### DETAILED DESCRIPTION

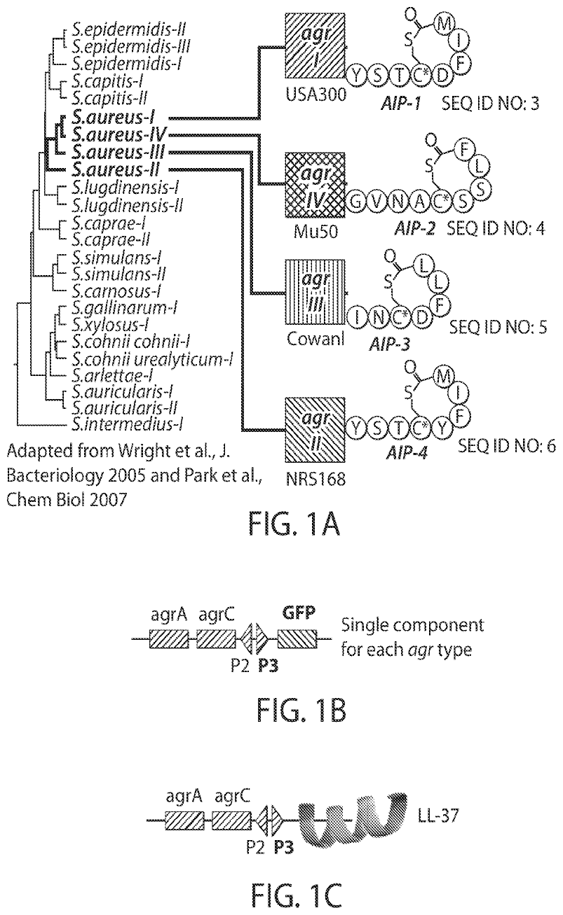

The design of the therapeutic biosensors of present disclosure is based on quorum sensing, a mechanism for cell-cell communication within a species. Staphylococcus (S.) aureus produces a chemical called an autoinducer peptide (AIP), which can be sensed by a transmembrane protein (AgrC to activate downstream processes within the cell via signal transducer AgrA (see, e.g., FIG. 1 of Novick R P et al. Annu. Rev. Genet. 2008 42: 541-64). S. aureus quorum sensing machinery has been classified into four major agr ‘Types,’ each of which is comprised of a different virulent strain. The therapeutic biosensors provided herein, in some embodiments, are based on skin commensal S. epidermidis strains (see, e.g., Olson M E et al. J Bacteriol. 2014 October; 196(19): 3482-3493) engineered to express each type of S. aureus AgrC and AgrA pair. These strains are able to sense the presence of S. aureus in the environment and turn on genes placed under the control of the P3 promoter, which responds to the corresponding type of AIP stimulus of AgrC/AgrA signal transduction.

### Therapeutic Biosensor Compositions and Cells

Some aspects of the present disclosure provide a composition comprising a commensal bacterial cell, such as an S. epidermidis cell (e.g., an agr-deficient S. epidermidis cell) comprising an engineered nucleic acid comprising a bidirectional promoter operably linked in one orientation to an S. aureus agrA coding sequence and an S. aureus agrC coding sequence and operably linked in another orientation to an antimicrobial peptide coding sequence, wherein the bidirectional promoter is activated by an S. aureus AgrA protein.

Other aspects provide a commensal bacterial cell, such as an S. epidermidis cell (e.g., an agr-deficient S. epidermidis cell) comprising an engineered nucleic acid comprising a bidirectional promoter operably linked in one orientation to an S. aureus agrA coding sequence and an S. aureus agrC coding sequence and operably linked in another orientation to an antimicrobial peptide coding sequence, wherein the bidirectional promoter is activated by an S. aureus AgrA protein.

Staphylococci are common bacterial colonizers of the skin and mucous membranes of humans and other mammals (Kloos W, Schleifer K H. In: Bergey's Manual of Systematic Bacteriology. PHA S, S M, ME S, JG H, editors. Baltimore: Williams & Wilkins; 1986). S. epidermidis belongs to the group of coagulase-negative staphylococci (CoNS), which is distinguished from coagulase-positive staphylococci such as S. aureus by lacking the enzyme coagulase. The species shows a high degree of diversity with 74 identified sequence types (STs) (Miragaia M et al. J Bacteriol 2007 March; 189(6):2540-52). Most isolates belong to clonal complex (CC) 2, which comprises the most frequently isolated ST2. Possibly, the successful spread of ST2 may be due to the fact that all ST2 isolates contain IS256 insertion sequences and ica genes (Li M. et al. J Med Microbiol. 2009 April; 58(Pt 4):456-461), two factors found correlated with S. epidermidis invasiveness (Faurschou M, et al. Microbes Infect. 2003; 5:1317-1327; Yao Y, et al. J Infect Dis. 2005; 191:289-298; Khardori N, et al. J Ind Microbiol. 1995; 15:148-151; Duguid I G, et al. Antimicrob Chemother. 1992; 30:803-810). In addition, most ST2 isolates show in vitro capacity to form biofilms (Li M, et al. J Med Microbiol. 2009; 58:456-461.). Genome information is available for several strains of S. epidermidis, including the biofilm-negative ATCC12228 (Zhang Y Q, et al. Mol Microbiol. 2003; 49:1577-1593), the biofilm-positive clinical isolate RP62A (Rogers K L, et al. Infect Dis Clin North Am. 2009; 23:73-98), and Tu3298, known for its production of epidermin and absence of the icaABCD operon (Moran J C et al. Genome Announc. 2016 March-April; 4(2): e00112-16). In some embodiments, the S. epidermidis cell is classified as strain Ti3298.

The S. epidermidis cells provided herein, in some embodiments, are agr-deficient. An S. epidermidis cell is herein considered agr-deficient if it does not express the AgrA or the AgrC protein. See, e.g., Vuong C. et al. Infect Immun. 2000 March; 68(3): 1048-1053. In some embodiments, the entire agr operon (encoding AgrBDCA) is inactivated (e.g., modified or deleted).

Methicillin-resistant Staphylococcus aureus (MRSA) is a multi-antibiotic resistant Gram-positive Staphylococci (Carrel, M., Perencevich, E. N., and David, M. Z. (2015) USA300 Methicillin-Resistant Staphylococcus aureus, United States, 2000-2013. Emerging Infect. Dis. 21, 1973) associated with severe skin infection in medical settings that can lead to sepsis and death (Lowy, F. N. Engl. J. Med. 1998; 339: 520). The frequency of MRSA-related deaths has surpassed those caused by HIV and is classified by the Centre for Disease Control (CDC) as a serious public health threat (Gupta, A K et al. Int. J. Dermatol. 2015; 54, 1226-32). The Gram-positive S. aureus uses a peptide-based two-component quorum-sensing system, known as Agr quorum sensing (agrQS) to regulate the expression of many virulence genes and biofilm formation, for controlling the microbial motile and sessile lifestyle (Gupta, R. et al. Proc. Natl. Acad. Sci. U.S.A 2015; 112 (45): 14036-41). This system, regulated by two promoters P2 and P3, encodes for the divergent transcriptional units RNAII and RNAIII, respectively. RNAII transcript encodes for all four genes agrB, agrD, agrC, and agrA, while RNAIII is the intracellular effector molecule that controls the Agr targets, including the expression of virulence factors such as alpha-toxin and delta-hemolysin (Otto, M. et al. FEMS Microbiol. Lett. 2015; 241: 135). AgrD is the pro-peptide that forms the quorum sensing molecule autoinducer peptide (AIP), while AgrB is a transmembrane protein that is responsible for cyclizing AgrD, generating AIP. AgrC and AgrA form the two-component system responsible for AIP detection and response relay, respectively (Otto, M. et al. 2015). Upon binding to AIP, AgrC, a transmembrane histidine kinase receptor, facilitates the phosphorylation of the transcriptional activator AgrA. The phosphorylated AgrA in turn activates gene expression regulated by P2 and P3 promoters producing a positive feedback loop of AIP production and activating various downstream virulence factors (Koenig, R. L., et al. J. Bacteriol. 2004; 186, 7549-55). There are four types of AgrC encoded by different Staphylococcus (Type I-IV) that specifically detects AIP Type I-IV, respectively (Geisinger, E., et al. J. Biol. Chem. 2008; 283 (14): 8930-38).

In some embodiments, the S. aureus agrA coding sequence and the S. aureus agrC coding sequence are classified as S. aureus Type I coding sequences. In some embodiments, the S. aureus agrA coding sequence and the S. aureus agrC coding sequence are classified as S. aureus Type II coding sequences. In some embodiments, the S. aureus agrA coding sequence and the S. aureus agrC coding sequence are classified as S. aureus Type III coding sequences. In some embodiments, the S. aureus agrA coding sequence and the S. aureus agrC coding sequence are classified as S. aureus Type IV coding sequences.

### Engineered Nucleic Acids

Also provided herein are engineered nucleic acids comprising a bidirectional promoter operably linked in one orientation to an S. aureus agrA coding sequence and an S. aureus agrC coding sequence and operably linked in another orientation to an antimicrobial peptide coding sequence, wherein the bidirectional promoter is activated by an S. aureus AgrA protein.

An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid introduced into an S. epidermidis includes a S. aureus nucleotide sequence.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). A gene, in some embodiments, includes a promoter sequence, coding regions (e.g., exons), non-coding regions (e.g., introns), and regulatory regions (also referred to as regulatory sequences). A promoter is a nucleotide sequence to which RNA polymerase binds to initial transcription (e.g., ATG). Promoters are typically located directly upstream from (at the 5′ end of) a transcription initiation site. A coding sequence (or other nucleotide sequence) is considered to be operably linked to a promoter if that promoter regulates transcription of the coding sequence.

Bidirectional promoters are short (<1 kbp) intergenic regions of DNA between the 5′ ends of the genes in a bidirectional gene pair. A “bidirectional gene pair” refers to two adjacent genes coded on opposite strands, with their 5′ ends oriented toward one another. An example of a bidirectional promoter is the S. aureus P2/P3promoter depicted in FIG. 1C, which shows the P2 promoter region operably linked to a cassette comprising agrC and agrA coding sequences and the P3 region operably linked to an LL-37 coding sequence. A bidirectional promoter is considered to be activated by an S. aureus AgrA protein if transcription of the nucleic acid to which the promoter is operably linked is initiated upon or following binding of the S. aureus AgrA protein to the promoter.

In some embodiments, a bidirectional promoter comprises a constitutive promoter. A constitutive promoter is an unregulated promoter that enables continual transcription of an associated nucleotide sequence. In some embodiment, a constitutive promoter is a constitutive S. epidermidis promoter. For example, a constitutive promoter may be a Staphylococcal accessory regulator A (SarA) promoter. Other constitutive promoters are contemplated herein.

In other embodiments, a bidirectional promoter comprises an S. aureus P2 promoter (e.g., S. aureus USA300 P2 promoter; ATTTAACAGTTAAGTATTTATTTCCTACAGTTAGGCAA TATAATGATAAAAGATTGTACTAAATCGTATAATGACAGTGAG (SEQ ID NO: 1)). A bidirectional promoters provided herein, in some embodiments, comprise an S. aureus P3 promoter (S. aureus USA300 P3 promoter: AAATTACAGTTAAGAATAAAAAACGACTA GTTAAGAAAAATTGGAAAATAAATGCTTTTAGCATGTTTTAATATAACTAG (SEQ ID NO: 2)). See, e.g., Rajasree K. et al. Biochemistry and Biophysics Reports; 6: 124-134.

A constitutive S. epidermidis promoter or the S. aureus P2 promoter, in some embodiments, is operably linked to an S. aureus agrA coding sequence and an S. aureus agrC coding sequence, while an S. aureus P3 promoter is operably linked to an antimicrobial peptide coding sequence. The S. aureus agrC coding sequence, in some embodiments, is located upstream (5′) from and in-frame with the S. aureus agrA coding sequence such that activation of the constitutive promoter or the P2 promoter, for example, results in transcription of both the S. aureus agrC and the S. aureus agrA coding sequences. In other embodiments, the S. aureus agrA coding sequence is located upstream (5′) from and in-frame with the S. aureus agrC coding sequence.

In some embodiments, the engineered nucleic acid is integrated into the S. epidermidis cell genome. Integration refers to a process that mediates the insertion of foreign nucleic acid (e.g., an engineered nucleic acid) into a chromosome, or another replicon, in order to form a covalently linked nucleic acid continuous with the host nucleic acid. In other embodiments, the engineered nucleic acid maintained episomally in the S. epidermidis cell.

An S. epidermidis cell, in some embodiments, includes a single copy of the engineered nucleic acid. In other embodiments, an S. epidermidis cell includes multiple copies (e.g., 2 or more, such as 2-10, 2-15, or 2-20 copies) of the engineered nucleic acid.

In some embodiments, the S. aureus agrA coding sequence is codon optimized for expression in S. epidermidis. In some embodiments, the S. aureus agrC coding sequence is codon optimized for expression in S. epidermidis. In some embodiments, the antimicrobial peptide coding sequence is codon optimized for expression in S. epidermidis. Codon optimization refers to experimental approaches designed to improve the codon composition of a recombinant gene based on various criteria without altering the amino acid sequence. See, e.g., Frelin L. et al. Gene Ther. 2004 March; 11(6):522-33.

### Antimicrobial Peptides

Many antimicrobial peptides (AMPs) display broad-spectrum and potent antimicrobial efficacy against bacteria, fungi and viruses. They are capable of being applied to treat various microbes and even drug-resistant ones. AMPs exist in various organisms (bacteria, fungi, animals and plants) and thousands of AMPs have been discovered and demonstrated (Jenssen H, et al. Clin Microbiol Rev. 2006 July; 19(3):491-511; and Kosciuczuk E M, et al. Mol Biol Rep. 2012 December; 39(12):10957-70). Of them, most are cationic AMPs that play the key antimicrobial roles. See, e.g., Lei J, et al. Am J Transl Res. 2019; 11(7): 3919-3931; Bechinger B et al., J Dent Res. 2017 March; 96(3):254-260; and Nuti R. et al., Curr Med Chem. 2017; 24(38):4303-4314 for non-limiting examples of AMPs, each of which is incorporated by reference herein.

Non-limiting examples of antimicrobial peptides that may be used as provided herein include LL-37, elafin, hiracin, BMAP-27, Fowlicidin-1, Fawlicidin-2, Fowlicidin-3, α-defensin 1, α-defensin 2, α-defensin 3, α-defensin 4, α-defensin 5, α-defensin 6, β-defensin 1, β-defensin 2, β-defensin 3, β-defensin 4, Defensin-like peptide-2, protegrin-3, antifungal heliomicin, and sugarcane defensin 5. Non-limiting examples of antimicrobial peptide drugs approved by Food and Drug Administration (FDA) include bacitracin, dalbavancin, daptomycin, enfuvirtide, oritavancin, teicoplanin, telaprevir, telavancin, and vancomycin. Other antimicrobial peptides may be used.

In some embodiments, the size of an antimicrobial peptide is 5 kD to 100 kD. For example, the size of an antimicrobial peptide may be 5 kD to 75 kD, 5 kD to 50 kD, 5 kD to 25 kD, 5 kD to 15 kD, or 5 kD to 10 kD. In some embodiments, the size of an antimicrobial peptide is 5, 6, 7, 8, 9, 10, 11, 12, 13, 14, 15, 20, 25, 30, 35, 40, 45, 50, 55, 60, 65, 70, 75, 80, 85, 90, 95, or 100 kD.

In some embodiments, the antimicrobial peptide is LL-37. The single human cathelicidin peptide LL-37 has been shown to have antimicrobial and anti-biofilm activity against multiple Gram-positive and Gram-negative human pathogens and have wound-healing effects on the host. The combination of the anti-biofilm effect and wound-healing properties of LL-37 may make it highly effective in resolving polymicrobially infected wounds when topically applied. See, e.g., Duplantier A J, et al. Immunol., 3 Jul. 2013.

In some embodiments, the antimicrobial peptide is elafin. Elafin is an endogenous human protein composed of an N-terminal transglutaminase substrate motif and a C-terminal WAP (whey acidic protein)-domain with antiproteolytic properties. Elafin is expressed predominantly in epithelial tissue and potently inhibits the neutrophil-derived serine proteases elastase and proteinase-3 by a competitive tight-binding mechanism. See, e.g., Shaw, L. et al. Biochem Soc Trans 2011 October; 39(5):1450-4.

In some embodiments, the antimicrobial peptide is hiracin. Hiracin is a Sec-dependent bacteriocin produced by Enterococcus hirae. Bacteriocins are ribosomally synthesized peptides or proteins with antimicrobial activity, and considerable research interest has focused on the bacteriocins produced by lactic acid bacteria (LAB) because of their potential applications in food, pharmaceuticals, nutraceuticals, and veterinary and human medicine. See, e.g., Hassan, M. et al. Adv Pharm Bull. 2015 September; 5(3): 393-401.

Also contemplated herein are antimicrobial peptides to which S. epidermidis cells are immune. That is, exposure of the S. epidermidis cell to the antimicrobial peptide expressed by the S. epidermidis cell does not kill the S. epidermidis cell. For example S. epidermidis may be outside the spectrum of inhibition of the antimicrobial peptide. As another example, the S. epidermidis cell may comprise a gene that confers resistance to the antimicrobial peptide. In some embodiments, an epidermin-resistance gene is introduced to an S. epidermidis cell (e.g., a Ti3298 cell).

In some embodiments, the antimicrobial peptide is linked to a secretion signal sequence (e.g., a signal peptide). A secretion signal sequence is a short amino acid sequence (e.g., 15-30 amino acids long) present at the N-terminus of the majority of newly synthesized proteins that are destined towards the secretory pathway (see, e.g., Rapoport T. Nature. 2007; 450 (7170): 663-9).

### Therapeutic Biosensor Cocktails

The present disclosure provides, in some aspects, “cocktail” compositions that include S. epidermidis cells engineered to respond to more than one type of S. aureus strain. Such a composition may comprise, for example, (a) an agr-deficient S. epidermidis cell comprising an engineered nucleic acid comprising a bidirectional promoter (i) operably linked in one orientation to an S. aureus agrA coding sequence and an S. aureus agrC coding sequence of a first strain and (ii) operably linked in another orientation to an antimicrobial peptide coding sequence, wherein the bidirectional promoter is activated by an S. aureus AgrA protein of the first strain; and (b) an agr-deficient S. epidermidis cell comprising an engineered nucleic acid comprising a bidirectional promoter (i) operably linked in one orientation to an S. aureus agrA coding sequence and an S. aureus agrC coding sequence of a second strain and (ii) operably linked in another orientation to an antimicrobial peptide coding sequence, wherein the bidirectional promoter is activated by an S. aureus AgrA protein of the second strain.

The first strain, in some embodiments, is a Type I S. aureus strain. The second strain, in some embodiments, is a Type II, Type III, or Type IV S. aureus strain. In some embodiments, the first strain is a Type I S. aureus strain, and the second strain is a Type II S. aureus strain. In other embodiments, the first strain is a Type I S. aureus strain, and the second strain is a Type III S. aureus strain. In yet other embodiments, the first strain is a Type I S. aureus strain, and the second strain is a Type III S. aureus strain. In still other embodiments, the first strain is a Type I S. aureus strain, and the second strain is a Type IV S. aureus strain.

In some embodiments, a composition comprises: (a) an agr-deficient S. epidermidis cell comprising an engineered nucleic acid comprising a bidirectional promoter (i) operably linked in one orientation to a Type I S. aureus agrA coding sequence and a Type I S. aureus agrC coding sequence and (ii) operably linked in another orientation to an antimicrobial peptide coding sequence, wherein the bidirectional promoter is activated by an S. aureus Type I AgrA protein; and (b) an agr-deficient S. epidermidis cell comprising an engineered nucleic acid comprising a bidirectional promoter (i) operably linked in one orientation to a Type II S. aureus agrA coding sequence and a Type II S. aureus agrC coding sequence and (ii) operably linked in another orientation to an antimicrobial peptide coding sequence, wherein the bidirectional promoter is activated by an S. aureus Type II AgrA protein.

In other embodiments, a composition comprises: (a) an agr-deficient S. epidermidis cell comprising an engineered nucleic acid comprising a bidirectional promoter (i) operably linked in one orientation to a Type I S. aureus agrA coding sequence and a Type I S. aureus agrC coding sequence and (ii) operably linked in another orientation to an antimicrobial peptide coding sequence, wherein the bidirectional promoter is activated by an S. aureus Type I AgrA protein; and (b) an agr-deficient S. epidermidis cell comprising an engineered nucleic acid comprising a bidirectional promoter (i) operably linked in one orientation to a Type III S. aureus agrA coding sequence and a Type III S. aureus agrC coding sequence and (ii) operably linked in another orientation to an antimicrobial peptide coding sequence, wherein the bidirectional promoter is activated by an S. aureus Type III AgrA protein.

In yet other embodiments, a composition comprises: (a) an agr-deficient S. epidermidis cell comprising an engineered nucleic acid comprising a bidirectional promoter (i) operably linked in one orientation to a Type I S. aureus agrA coding sequence and a Type I S. aureus agrC coding sequence and (ii) operably linked in another orientation to an antimicrobial peptide coding sequence, wherein the bidirectional promoter is activated by an S. aureus Type IV AgrA protein; and (b) an agr-deficient S. epidermidis cell comprising an engineered nucleic acid comprising a bidirectional promoter (i) operably linked in one orientation to a Type IV S. aureus agrA coding sequence and a Type IV S. aureus agrC coding sequence and (ii) operably linked in another orientation to an antimicrobial peptide coding sequence, wherein the bidirectional promoter is activated by an S. aureus Type IV AgrA protein.

In some embodiments, a composition comprises: (a) an agr-deficient S. epidermidis cell comprising an engineered nucleic acid comprising a bidirectional promoter (i) operably linked in one orientation to a Type II S. aureus agrA coding sequence and a Type II S. aureus agrC coding sequence and (ii) operably linked in another orientation to an antimicrobial peptide coding sequence, wherein the bidirectional promoter is activated by an S. aureus Type II AgrA protein; and (b) an agr-deficient S. epidermidis cell comprising an engineered nucleic acid comprising a bidirectional promoter (i) operably linked in one orientation to a Type III S. aureus agrA coding sequence and a Type III S. aureus agrC coding sequence and (ii) operably linked in another orientation to an antimicrobial peptide coding sequence, wherein the bidirectional promoter is activated by an S. aureus Type III AgrA protein.

In other embodiments, a composition comprises: (a) an agr-deficient S. epidermidis cell comprising an engineered nucleic acid comprising a bidirectional promoter (i) operably linked in one orientation to a Type II S. aureus agrA coding sequence and a Type II S. aureus agrC coding sequence and (ii) operably linked in another orientation to an antimicrobial peptide coding sequence, wherein the bidirectional promoter is activated by an S. aureus Type II AgrA protein; and (b) an agr-deficient S. epidermidis cell comprising an engineered nucleic acid comprising a bidirectional promoter (i) operably linked in one orientation to a Type IV S. aureus agrA coding sequence and a Type IV S. aureus agrC coding sequence and (ii) operably linked in another orientation to an antimicrobial peptide coding sequence, wherein the bidirectional promoter is activated by an S. aureus Type IV AgrA protein.

In yet other embodiments, a composition comprises: (a) an agr-deficient S. epidermidis cell comprising an engineered nucleic acid comprising a bidirectional promoter (i) operably linked in one orientation to a Type III S. aureus agrA coding sequence and a Type III S. aureus agrC coding sequence and (ii) operably linked in another orientation to an antimicrobial peptide coding sequence, wherein the bidirectional promoter is activated by an S. aureus Type III AgrA protein; and (b) an agr-deficient S. epidermidis cell comprising an engineered nucleic acid comprising a bidirectional promoter (i) operably linked in one orientation to a Type IV S. aureus agrA coding sequence and a Type IV S. aureus agrC coding sequence and (ii) operably linked in another orientation to an antimicrobial peptide coding sequence, wherein the bidirectional promoter is activated by an S. aureus Type IV AgrA protein.

### Formulations

The compositions herein may be formulated for topical administration. For example, a composition may be formulated as a biofilm, a cream, a lotion, or a gel. A composition is considered to be “formulated for topical administration” if it comprises an excipient suitable for application to human skin.

A formulation comprising a bacterial cell, for example, may comprise any amount of the engineered microbe to produce a therapeutically effective amount of a recombinant polypeptide. In some embodiments, a formulation comprises at least 0.01%, 0.05%, 0.1%, 0.2%, 0.3%, 0.4%, 0.5%, 0.6%, 0.7%, 0.8%, 0.9%, 1.0%, 1.5%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 25.0%, 30.0%, 35.0%, 40.0%, 45.0%, 50.0% or more by weight of the engineered microbe. In some embodiments, the upper limit is 90.0% by weight of engineered microbe.

In some embodiments, a formulation comprises at least 0.01% to 30%, 0.01% to 20%, 0.01% to 5%, 0.1% to 30%, 0.1% to 20%, 0.1% to 15%, 0.1% to 10%, 0.1% to 5%, 0.2% to 5%, 0.3% to 5%, 0.4% to 5%, 0.5% to 5%, 1% to 5%, or more by weight of the engineered microbe.

Dosage forms for the topical or transdermal administration include powders, sprays, ointments, pastes, creams, lotions, gels, solutions, patches, drops and inhalants. A bacterial cell and/or a recombinant polypeptide may be mixed under sterile conditions with a suitable pharmaceutically-acceptable excipient (e.g., diluent and/or carrier). Thus, ointments, pastes, creams and gels may contain excipients. Powders and sprays may contain excipients and/or propellants.

In some embodiments, a formulation is a topical formulation. A topical formulation may be, for example, in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. In some embodiments, a formulation can include a living cell culture composition and can comprise at least one engineered bacterial strain that produces a therapeutically effective recombinant polypeptide. This engineered living cell culture composition may deliver the recombinant polypeptide, for example, directly to the skin for treating or preventing abnormal skin conditions.

Topical formulations include those in which any other active ingredient(s) is (are) dissolved or dispersed in a dermatological vehicle known in the art (e.g., aqueous or nonaqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g., a mineral oil such as a liquid paraffin, natural or synthetic triglycerides such as MIGLYOL™, or silicone oils, such as dimethicone). Depending, inter alia, upon the nature of the formulation as well as its intended use and site of application, the formulation used can contain at least one component (for example, when the formulation is an aqueous gel, components in addition to water) selected from the following list: a solubilizing agent or solvent (e.g., a O-cyclodextrin, such as bydroxypropyl O-cyclodextrin, or an alcohol or polyol such as ethanol, propylene glycol or glycerol); a thickening agent (e.g., hydroxyethylceliulose, hydroxypropylcellulose, carboxymethylcellulose or carbomer); a gelling agent (e.g., a polyoxyethylene-polyoxypropylene copolymer); a preservative (e.g., benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A pharmaceutically acceptable excipient can also be incorporated in a formulation and can be any excipient (e.g., carrier) conventionally used in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art

A film former, when it dries, forms a protective film over the site of application. The film inhibits removal of the active ingredient and keeps it in contact with the site being treated. An example of a film former that is suitable for use herein is Flexible Collodion, USP. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction and are typically liquid or semiliquid preparations in which particles, including the active agent, are present in a water or alcohol base. Lotions are usually suspensions of solids, and in some embodiments, comprise a liquid oily emulsion of the oil-in-water type. Lotions may be used for treating large body areas, because of the ease of applying a more fluid composition. Lotions, in some embodiments, contain suspending agents to produce better dispersions as well as compounds useful for localizing and holding the active agent in contact with the skin, e.g., methylcellulose, sodium carboxymethylcellulose, or the like.

Solutions are homogeneous mixtures prepared by dissolving one or more chemical substances (solutes) in a liquid such that the molecules of the dissolved substance are dispersed among those of the solvent. A solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Example organic macromolecules, e.g., gelling agents, are cross-linked acrylic acid polymers, such as the carbomer family of polymers, e.g., carboxypolyalkylenes that can be obtained commercially under the CARBOPOL® trademark. Other examples are hydrophilic polymers, such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthaiate, and methylcellulose; gums, such as tragacanth and xanthan gum; sodium alginate; and gelatin. In order to prepare a uniform gel, dispersing agents, such as alcohol or glycerin, can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy, for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as plasticizing enhancers, e.g., enhancers that have a molecular weight in the range of 150 to 1000, an aqueous solubility of less than 1 wt. %, less than 0.5 wt. %, or less than 0.2 wt. %. The Hildebrand solubility parameter (6) of plasticizing enhancers is in the range of 2.5 to 10, or in the range of 5 to 10. Examples of lipophilic enhancers include fatty esters, fatty alcohols, and fatty ethers. Examples of specific fatty acid esters include methyl laurate, ethyl oleate, propylene glycol propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995).

Various other additives can be included in the compositions of the present disclosure in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in formulations of the present disclosure are as follows: preservatives, such as sorbate; solvents, such as isopropanol and propylene glycol; astringents, such as menthol and ethanol; emollients, such as polyalkylene methyl glucosides; humectants, such as glycerine; emulsifiers, such as glycerol stearate, PEG-100 stearate, polyglyceryl-3 hydroxylauryl ether, and polysorbate 60; sorbitol and other polyhydroxyalcohols, such as polyethylene glycol; sunscreen agents, such as octyl methoxyl cinnamate (available commercially as Parsol MCX) and butyl methoxy benzoylmethane (available under the tradename PARSOL® 1789); antioxidants such as ascorbic acid (vitamin C), a-tocopherol (Vitamin E), β-tocopherol, γ-tocopherol, δ-tocopherol, ε-tocopherol, ζι-tocopherol, Z^{Γ}-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the CARBOPOL® trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants, and/or pharmaceutically acceptable salts, esters, amides, or other derivatives thereof. Additional agents include those that are capable of improving oxygen supply in skin tissue. Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene.

Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from 0.01% to 20% of the total weight of the topical formulation.

Formulations of the present disclosure can also include conventional additives such as opacifiers, fragrance, colorant, stabilizers, surfactants, and the like. In some embodiments, other agents can also be added, such as antimicrobial agents, to prevent spoilage upon storage, i.e., to inhibit growth of microbes such as yeasts and molds.

Suitable antimicrobial agents are typically selected from the group consisting of the methyl and propyl esters of p-hydroxybenzoic acid (methyl and propyl paraben), sodium benzoate, sorbic acid, imidurea, and combinations thereof. In other embodiments, other agents can also be added, such as repressors and inducers, e.g., to inhibit (glycose) or induce (xylose) the production of the polypeptide of interest. Such additives can be employed provided they are compatible with and do not interfere with the function of the formulations.

Formulations can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than 20 wt. %, more typically not more than 5 wt. %, of the formulation.

Further suitable pharmacologically active agents that can be incorporated into the present formulations in some embodiments and thus topically applied along with the engineered microbe and/or recombinant polypeptide include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment but will normally involve one or more applications per day on an ongoing basis. One of ordinary skill can readily determine the optimum amount of the formulation to be administered, administration methodologies and repetition rates. In general, it is contemplated that the formulations of the invention will be applied in the range of once or twice weekly up to once or twice daily.

### Therapeutic/Prophylactic Methods

The compositions and cells provided herein may be used to treat or prevent a bacterial cell infection.

Some aspects of the present disclosure provide a method comprising administering to a subject a composition comprising an agr-deficient Staphylococcus (S.) epidermidis cell comprising an engineered nucleic acid comprising a bidirectional promoter operably linked in one orientation to an S. aureus agrA coding sequence and an S. aureus agrC coding sequence and operably linked in another orientation to an antimicrobial peptide coding sequence, wherein the bidirectional promoter is activated by an S. aureus AgrA protein.

A subject, in some embodiments, is a human subject. In some embodiments, the subject has a virulent bacterial infection, such as a S. aureus infection. Virulence is an ability of an organism, such as bacteria, to infect a host, such as a human, and cause a disease. The S. aureus infection, in some embodiments, is a Methicillin-resistant Staphylococcus aureus (MRSA) infection. MRSA a cause of S. aureus (staph) infection that is difficult to treat because of resistance to some antibiotics. This type of bacteria is often present on the skin. While S. aureus is often harmless, it can cause serious infections that can lead to sepsis or death.

Common skin infections that may be treated as provided herein include cellulitis, erysipelas, impetigo, folliculitis, and furuncles and carbuncles. Other skin infections may be treated using the compositions and/or cells of the present disclosure.

In some embodiments, a method comprises administering to a subject a composition comprising an agr-deficient S. epidermidis cell comprising an engineered nucleic acid comprising a bidirectional promoter operably linked in one orientation to an S. aureus agrA coding sequence and an S. aureus agrC coding sequence and operably linked in another orientation to a LL-37 peptide coding sequence, wherein the bidirectional promoter is activated by an S. aureus AgrA protein.

In some embodiments, a method comprises administering to a subject a composition comprising an agr-deficient S. epidermidis cell comprising an engineered nucleic acid comprising a bidirectional promoter operably linked in one orientation to an S. aureus agrA coding sequence and an S. aureus agrC coding sequence and operably linked in another orientation to an elafin peptide coding sequence, wherein the bidirectional promoter is activated by an S. aureus AgrA protein.

In some embodiments, a method comprises administering to a subject a composition comprising an agr-deficient S. epidermidis cell comprising an engineered nucleic acid comprising a bidirectional promoter operably linked in one orientation to an S. aureus agrA coding sequence and an S. aureus agrC coding sequence and operably linked in another orientation to a hiracin peptide coding sequence, wherein the bidirectional promoter is activated by an S. aureus AgrA protein.

In some embodiments, a method comprises administering to a subject an agr-deficient S. epidermidis cell comprising an engineered nucleic acid comprising a bidirectional promoter operably linked in one orientation to an S. aureus agrA coding sequence and an S. aureus agrC coding sequence and operably linked in another orientation to an antimicrobial peptide coding sequence, wherein the bidirectional promoter is activated by an S. aureus AgrA protein.

In some embodiments, a method comprises administering to a subject an engineered nucleic acid comprising a bidirectional promoter operably linked in one orientation to an S. aureus agrA coding sequence and an S. aureus agrC coding sequence and operably linked in another orientation to a LL-37 coding sequence, wherein the bidirectional promoter is activated by an S. aureus AgrA protein.

In some embodiments, a method comprises administering to a subject an engineered nucleic acid comprising a bidirectional promoter operably linked in one orientation to an S. aureus agrA coding sequence and an S. aureus agrC coding sequence and operably linked in another orientation to a elafin, coding sequence, wherein the bidirectional promoter is activated by an S. aureus AgrA protein.

In some embodiments, a method comprises administering to a subject an engineered nucleic acid comprising a bidirectional promoter operably linked in one orientation to an S. aureus agrA coding sequence and an S. aureus agrC coding sequence and operably linked in another orientation to a hiracin coding sequence, wherein the bidirectional promoter is activated by an S. aureus AgrA protein.

In some embodiments, a method comprises administering to a subject a composition comprising: (a) an agr-deficient S. epidermidis cell comprising an engineered nucleic acid comprising a bidirectional promoter (i) operably linked in one orientation to an S. aureus agrA coding sequence and an S. aureus agrC coding sequence of a first strain and (ii) operably linked in another orientation to an antimicrobial peptide coding sequence, wherein the bidirectional promoter is activated by an S. aureus AgrA protein of the first strain; and (b) an agr-deficient S. epidermidis cell comprising an engineered nucleic acid comprising a bidirectional promoter (i) operably linked in one orientation to an S. aureus agrA coding sequence and an S. aureus agrC coding sequence of a second strain and (ii) operably linked in another orientation to an antimicrobial peptide coding sequence, wherein the bidirectional promoter is activated by an S. aureus AgrA protein of the second strain.

In some embodiments, a method comprises administering to a subject a composition comprising: (a) an agr-deficient S. epidermidis cell comprising an engineered nucleic acid comprising a bidirectional promoter (i) operably linked in one orientation to a Type I S. aureus agrA coding sequence and a Type I S. aureus agrC coding sequence and (ii) operably linked in another orientation to an antimicrobial peptide coding sequence, wherein the bidirectional promoter is activated by an S. aureus Type I AgrA protein; and (b) an agr-deficient S. epidermidis cell comprising an engineered nucleic acid comprising a bidirectional promoter (i) operably linked in one orientation to a Type II, III, or IV S. aureus agrA coding sequence and a Type II, III, or IV S. aureus agrC coding sequence and (ii) operably linked in another orientation to an antimicrobial peptide coding sequence, wherein the bidirectional promoter is activated by an S. aureus Type II, III, or IV AgrA protein.

A composition and/or bacterial cell of the present disclosure, in some embodiments, is administered in a prophylactically effective amount or a therapeutically effective amount. The prophylactically effective amount, in some embodiments, is effective to prevent a bacterial cell infection. In some embodiments, the therapeutically effective amount is effective to treat a bacterial cell infection. The bacterial cell infection may be, for example, an S. aureus such as a MRSA infection.

With respect to prevention of a bacterial infection, it should be understood that a prophylactically effective amount of a cell or composition need not entirely eradicate the bacteria but should prevent the bacterial cell population present in the subject, for example, on the skin of the subject, from causing symptoms of a disease (e.g., fever, rash, nausea, etc.). In some embodiments, a prophylactically effective amount of a cell or composition reduces the bacterial cell population present in (or on the skin of) the subject by at least 30%, at least 40%, at least 50%, at least 60%, at least 70%, at least 80%, or at least 90%. Likewise, with respect to treatment of a bacterial infection, it should be understood that a therapeutically effective amount of a cell or composition need not cure a disease associated with a bacterial infection or entirely eradicate the bacterial cell population but should alleviate at least one symptom of the disease and reduce the bacterial cell population present in (or on the skin of) the subject by at least 30%, at least 40%, at least 50%, at least 60%, at least 70%, at least 80%, or at least 90%.

### EXAMPLES

### Example 1: Biosensor Development

Because it is advantageous for the S. epidermidis biosensor strains to sense all four agr types of S. aureus, three different versions of the sensor locus were engineered and incorporated into S. epidermidis (three versions because type III can detect both III and IV AIP) (FIGS. 1B, 1C). An initial design created a “four-component” sensor, in which the AgrC/AgrC machinery of all four components were synthesized as a single construct. A “cocktail” of the three versions of the sensor locus, as well as a single Type I version of the sensor locus have also been designed, as Type I accounts for a majority of MRSA outbreak types.

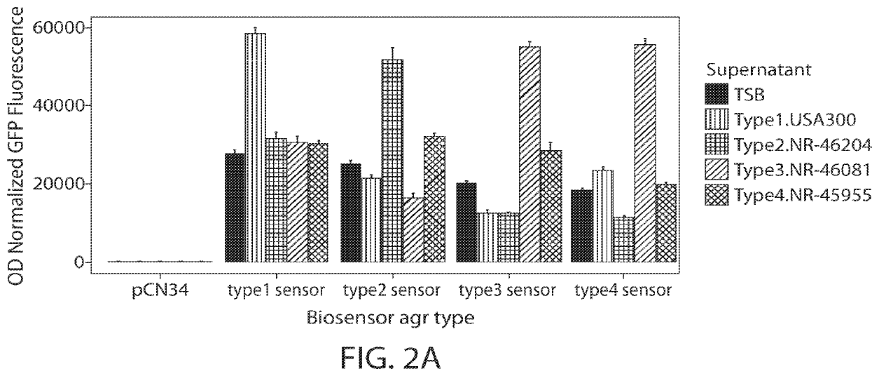

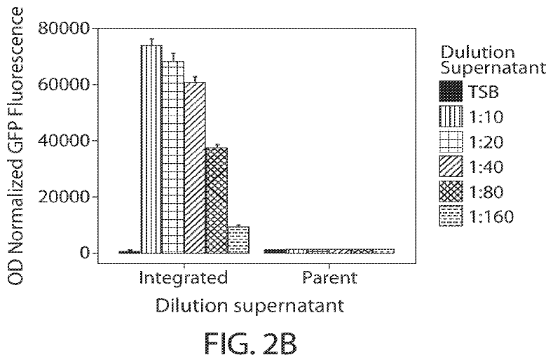

Initial validation of the biosensor construct was performed in a Staphylococcal shuttle plasmid vector transformed into an agr-deficient strain of S. epidermidis (Ti3298). Sensors from all four agr types were produced, tested, and characterized to determine specificity and sensitivity (FIG. 2A). In the presence of S. aureus supernatant, strong and specific induction of the biosensor expressing green fluorescent protein (GFP) was observed, with overlap between Types III and IV. Subsequently, genomic integrations of the biosensor constructs were created to circumvent a requirement for plasma maintenance. The genomic integrant showed similar strong dose-dependent activation of the Type I integrated biosensor in response to the amount of S. aureus Type I culture supernatant provided. The integrant also showed reduced background from basal expression of the biosensor, likely due to single-copy genomic expression versus a multi-copy plasmid (FIG. 2B).

Sensors from all four agr types are being tested in reconstructed human epidermis and mouse skin.

While any molecule can theoretically be placed under the control of the biosensor, an ideal molecule would be small in size and simple with respect to processing and modifications to facilitate expression and export. Other considerations for selecting an ideal molecule are immunity of the S. epidermidis strain for the molecule, ability to be secreted/transported, likelihood of acquired antimicrobial resistance in the target S. aureus, narrow spectrum of inhibition to avoid targeting of other commensal skin microbes, and the need for codon optimization for expression in S. epidermidis.

A list of over 100 potential candidates of known and hypothetical anti-S. aureus molecules were identified from the literature and narrowed to three microbial and human-derived candidates based on ideal characteristics including size and the availability of a resistance gene to protect the producer strain S. epidermidis (Table 1). The three candidates are: elafin (human origin), hiracin (Enterococcus hirae origin), and LL-37 (human origin).

TABLE 1

| | | | | Size of Peptide |
|---|---|---|---|---|
| Gene | Source | Immunity | Size of Peptide | Complex |
| Elafin | Human* | No | 61 aa; 6.6 kD | 11.4 kD |
| Hiracin | Enterococcus | Yes | 74 aa; 8.1 kD | 12.9 kD |
| | hirae | | | |
| LL-37 | Human* | No | 37 aa; 4.5 kD | 9.4 kD |

*codon optimized sequence

A gene encoding each of the three candidate molecules was cloned into an inducible tetracycline vector and transformed into S. epidermidis strain Ti3298 to evaluate basal activity. The AMP LL-37 was tested with several export signaling peptides. All three candidate molecules demonstrated strong anti-S. aureus activity (data not shown). Each of the three candidate molecules are being evaluated in the Type I biosensor in vitro and in vivo.

### Example 2. Biosensor Activity

S. aureus strains of four agr types are plated on TSA plates, respectively (10⁵ cells each). On each plate, S. epidermidis biosensor strains, featuring chromosomal integrants of an antimicrobial peptide under quorum sensing control of the different agr types are spotted. Two clones are used for each type, and each clone is spotted with two doses, 10⁴ and 10⁶ cells, respectively. The parent strain for integrations, S. epidermidis Tu3298 Δagr, is also spotted as a control with 10⁴ and 10⁶ cells, respectively. Halos around the spots indicate killing of the tester S. aureus strain, and cross-reactivity between biosensor agr types is tested.

### Example 3. In Vivo Efficacy Against MRSA

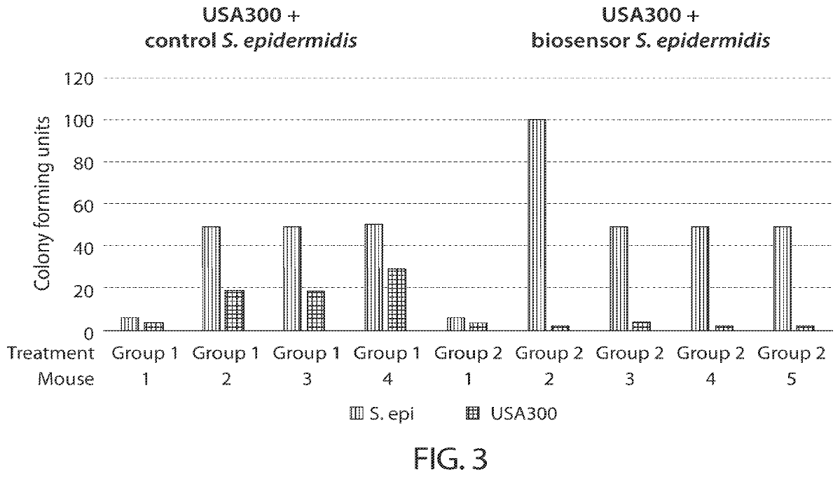

Experimental groups of germ-free C57BL/6J mice (n=4 or 5 each) were depilated and wounded with a 3×3 mm dorsal punch biopsy. A mixture of (1) 10⁵ USA300 (methicillin-resistant S. aureus (MRSA) agr type 1 strain)+10⁸ S. epidermidis Tu3298Δagr (parent S. epidermidis strain, no biosensor, Group 1) or (2) 10⁵ USA300+10⁸ S. epidermidis lysostaphin-producing biosensor (Group 2) was applied in petroleum jelly to the wounded area of each mouse. Bacteria were recovered after 48 hours with 1 cm² of skin around the punch and homogenized in 1 mL 1× phosphate buffered saline and diluted 1:10, 1:100, 1:1000, 1:10000, and 1:100000. 10 μL of the dilutions were plated onto mannitol salt agar plates to differentiate USA300 S. aureus from S. epidermidis, and the colony forming units were quantified. Bacterial identity is determined by colony size, color, and by MALDI-TOF. The number of S. aureus colony forming units (CFUs) was reduced in the presence of S. epidermidis lysostaphin-producing biosensor (FIG. 3). Thus, S. epidermidis expressing a biosensor is effective in treating MRSA in vivo.

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. An engineered Staphylococcus (S.) epidermidis cell comprising an S. aureus quorum-sensing receptor.

2. The engineered cell of claim 1, wherein the S. aureus quorum-sensing receptor is a component of an S. aureus quorum-sensing signal-response system.

3. The engineered cell of claim 1 or 2, wherein the cell is an agr deficient mutant S. epidermidis cell.

4. The engineered cell of claim 2 or 3, wherein the quorum-sensing signal-response system comprises a first promoter sequence operably linked to a therapeutic peptide coding sequence.

5. The engineered cell of claim 4, wherein the promoter sequence is activated by signaling of the quorum-sensing signal-response system.

6. The engineered cell of claim 4 or 5, wherein the therapeutic peptide coding sequence is an antimicrobial peptide coding sequence.

7. The engineered cell of any one of claims 2-6, wherein the quorum-sensing signal-response system comprises (a) an S. aureus agrA coding sequence encoding AgrA and (b) an S. aureus agrC coding sequence encoding S. aureus AgrC.

8. The engineered cell of claim 7, wherein the first promoter sequence is activated by S. aureus AgrA.

9. The engineered cell of claim 7 or 8, wherein the S. aureus agrA coding sequence is operably linked to a second promoter sequence.

10. The engineered cell of any one of claims 7-9, wherein the S. aureus agrC coding sequence is operably linked to a second promoter sequence.

11. The engineered cell of claim 7, wherein the S. aureus agrA coding sequence and the S. aureus agrC coding sequence are operably linked to a second promoter sequence.

12. The engineered cell of any one of claims 9-11, wherein the second promoter sequence is a constitutive promoter sequence.

13. The engineered cell of any one of claims 9-12, wherein the first promoter sequence and second promoter sequence are oriented in opposite directions relative to each other.

14. The engineered cell of claim 13, wherein the first promoter sequence and the second promoter sequence are components of a bidirectional promoter.

15. An engineered agr deficient mutant Staphylococcus (S.) epidermidis cell comprising a nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial peptide coding sequence, wherein the bidirectional promoter is activated by an S. aureus AgrA protein.

16. The engineered cell of claim any one of claims 6-15, wherein the size of the antimicrobial peptide is 5 kD to 100 kD, or 5 kD to 50 kD.

17. The engineered cell of any one of claims 6-16, wherein the antimicrobial peptide is LL-37.

18. The engineered cell of any one of claims 6-16, wherein the antimicrobial peptide is elafin.

19. The engineered cell of any one of claims 6-16, wherein the antimicrobial peptide is hiracin.

20. An engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial LL-37 peptide coding sequence, wherein the bidirectional promoter is activated by an S. aureus AgrA protein.

21. An engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial elafin peptide coding sequence, wherein the bidirectional promoter is activated by an S. aureus AgrA protein.

22. An engineered agr deficient mutant Staphylococcus (S.) epidermidis cell comprising an engineered nucleic acid comprising a bidirectional promoter, wherein the bidirectional promoter is (a) operably linked in one orientation to an S. aureus agrA coding sequence and an S. aureus agrC coding sequence and (b) operably linked in another orientation to an antimicrobial hiracin peptide coding sequence, wherein the bidirectional promoter is activated by an S. aureus AgrA protein.

23. The engineered cell of any one of claims 14-22, wherein the bidirectional promoter comprises a constitutive promoter sequence, optionally an S. epidermidis promoter sequence, and an S. aureus P3 promoter sequence, or an S. aureus P2 promoter sequence and an S. aureus P3 promoter sequence.

24. The engineered cell of claim 23, wherein the constitutive promoter sequence or the S. aureus P2 promoter sequence is operably linked to the S. aureus agrA coding sequence and the S. aureus agrC coding sequence, and the S. aureus P3 promoter sequence is operably linked to the antimicrobial peptide coding sequence.

25. The engineered cell of any one of claims 7-24, wherein the S. aureus agrA coding sequence and the S. aureus agrC coding sequence are classified as S. aureus Type I, Type II, Type III, or Type IV coding sequences.

26. The engineered cell of claim 25, wherein the S. aureus agrA coding sequence and the S. aureus agrC coding sequence are classified as S. aureus Type I coding sequences.

27. The engineered cell of any one of the preceding claims, wherein the S. epidermidis cell is classified as strain Tu3298 or an agr deficient mutant thereof.

28. The engineered cell of any one of the preceding claims, wherein the engineered nucleic acid is integrated into the S. epidermidis cell genome.

29. The engineered cell of claim 28, wherein the S. epidermidis cell genome includes a single copy of the engineered nucleic acid.

30. The engineered cell of any one of claims 7-29, wherein the S. aureus agrA coding sequence, the S. aureus agrC coding sequence, and/or the antimicrobial peptide coding sequence is codon optimized for expression in S. epidermidis.

31. The engineered cell of any one of the preceding claims, wherein the cell is immune to the antimicrobial peptide.

32. The engineered cell of claim 31, wherein the cell comprises a gene the confers resistance to the antimicrobial peptide.

33. The engineered cell of any one of claims 6-32, wherein the antimicrobial peptide is linked to a secretion signal sequence.

34. A composition comprising the engineered cell of any one of the preceding claims.

35. The composition of claim 34 formulated for topical administration.

36. The composition of claim 35 formulated as a biofilm, a cream, a lotion, or a gel.

37. An engineered nucleic acid comprising (a) a first promoter sequence operably linked to a LL-37 coding sequence and (b) a second promoter sequence operably linked to an S. aureus agrA coding sequence and an S. aureus agrC coding sequence, wherein the first promoter is activated by an S. aureus AgrA protein.

38. An engineered nucleic acid comprising (a) a first promoter sequence operably linked to an elafin coding sequence and (b) a second promoter sequence operably linked to an S. aureus agrA coding sequence and an S. aureus agrC coding sequence, wherein the first promoter is activated by an S. aureus AgrA protein.

39. An engineered nucleic acid comprising (a) a first promoter sequence operably linked to a hiracin coding sequence and (b) a second promoter sequence operably linked to an S. aureus agrA coding sequence and an S. aureus agrC coding sequence, wherein the first promoter is activated by an S. aureus AgrA protein.

40. The engineered nucleic acid of any one of claims 37-39, wherein the first promoter sequence and second promoter sequence are oriented in opposite directions relative to each other.

41. The engineered nucleic acid of claim 40, wherein the first promoter sequence and the second promoter sequence are components of a bidirectional promoter.

42. A composition comprising two cells, a first cell and a second cell, of any one of the preceding claims, wherein the S. aureus quorum-sensing signal-response system of the first cell is of a first strain, and the S. aureus quorum-sensing signal-response system of the second cell is of a second strain.

43. The composition of claim 42, wherein the first strain and the second strain are selected from a Type I S. aureus strain, a Type II S. aureus strain, a Type III S. aureus strain, and a Type IV S. aureus strain.

44. The composition of claim 43, wherein:
(a) the first strain is a Type I S. aureus strain, and the second strain is a Type II S. aureus strain;
(b) the first strain is a Type I S. aureus strain, and the second strain is a Type III S. aureus strain;
(c) the first strain is a Type I S. aureus strain, and the second strain is a Type IV S. aureus strain;
(d) the first strain is a Type II S. aureus strain, and the second strain is a Type III S. aureus strain;
(e) the first strain is a Type II S. aureus strain, and the second strain is a Type IV S. aureus strain; or
(f) the first strain is a Type III S. aureus strain, and the second strain is a Type IV S. aureus strain.

45. A composition comprising three cells, a first cell, a second cell, and a third cell, of any one of the preceding claims, wherein the S. aureus quorum-sensing signal-response system of the first cell is of a first strain, the S. aureus quorum-sensing signal-response system of the second cell is of a second strain, and the S. aureus quorum-sensing signal-response system of the third cell is of a third strain.

46. The composition of claim 45, wherein the first strain, the second strain, and the third strain are selected from a Type I S. aureus strain, a Type II S. aureus strain, a Type III S. aureus strain, and a Type IV S. aureus strain.

47. The composition of claim 46, wherein the first strain is a Type I S. aureus strain and
(a) the second strain is a Type II S. aureus strain, and the third strain is a Type III S. aureus strain;
(b) the second strain is a Type II S. aureus strain, and the third strain is a Type IV S. aureus strain; or
(c) the second strain is a Type III S. aureus strain, and the third strain is a Type IV S. aureus strain.

48. A composition comprising four cells, a first cell, a second cell, a third cell, and a fourth cell, of any one of the preceding claims, wherein the S. aureus quorum-sensing signal-response system of the first cell is of a first strain, the S. aureus quorum-sensing signal-response system of the second cell is of a second strain, the S. aureus quorum-sensing signal-response system of the third cell is of a third strain, and the S. aureus quorum-sensing signal-response system of the fourth cell is of a fourth strain,

49. The composition of claim 48, wherein the first strain, the second strain, the third strain, and the fourth strain are selected from a Type I S. aureus strain, a Type II S. aureus strain, a Type III S. aureus strain, and a Type IV S. aureus strain, and each cell is of a different strain relative to each other.

50. A method, comprising administering to a subject the composition of any one of the preceding claims.

51. The method of claim 50, wherein the subject has a virulent bacterial infection.

52. The method of claim 51, wherein the virulent bacterial infection is a bacterial skin infection.

53. The method of claim 51 or 52, wherein the virulent bacterial infection is a Methicillin-resistant Staphylococcus aureus (MRSA) infection.


SEQUENCE LISTING (6 sequences)

SEQ ID NO: 1 - DNA, 81 bp, Staphylococcus aureus
atttaacagttaagtatttatttcctacagttaggcaatataatgataaaagattgtactaaatcgtataatgacagtgag

SEQ ID NO: 2 - DNA, 80 bp, Staphylococcus aureus
aaattacagttaagaataaaaaacgactagttaagaaaaattggaaaataaatgcttttagcatgttttaatataactag

SEQ ID NO: 3 - protein, 8 aa, Staphylococcus aureus
YSTCDFIM

SEQ ID NO: 4 - protein, 9 aa, Staphylococcus aureus
GVNACSSLF

SEQ ID NO: 5 - protein, 7 aa, Staphylococcus aureus
INCDFLL

SEQ ID NO: 6 - protein, 8 aa, Staphylococcus aureus
YSTCYFIM